(19) United States
(12) Patent Application Publication
(10) Pub. No.: US 2016/0250064 A1
(43) Pub. Date: Sep. 1, 2016

(54) System for Preventing Pregnancy and Covertly Treating Erectile Dysfunction

(71) Applicant: William Andrew Ross, Naperville, IL (US)

(72) Inventor: William Andrew Ross, Naperville, IL (US)

(21) Appl. No.: 15/056,821

(22) Filed: Feb. 29, 2016

Related U.S. Application Data
(63) Continuation-in-part of application No. 29/521,314, filed on Mar. 23, 2015.
(60) Provisional application No. 62/126,145, filed on Feb. 27, 2015.

(51) Int. Cl. A61F 6/04; A61F 6/00

(52) U.S. Cl. CPC A61F 6/04; A61F 6/005

(57) ABSTRACT

A system for preventing pregnancy and covertly treating erectile dysfunction is used to help the user maintain an erection by constricting the penis of the user. The system includes a condom which, like a traditional condom, is used to capture semen in order to prevent pregnancy. The system includes a first constriction band, a second constriction band, and a constriction ring which each work to constrict areas of the penis to limit blood flow so that an erection may be maintained during sex. The condom includes a tubular body and a length-adjustable head. The tubular body helps to hold the length-adjustable head in place over the head of the penis. The length-adjustable head captures ejaculated semen and also helps to keep the first constriction band in place.

The current application claims a priority to the U.S. Provisional Patent application Ser. No. 62/126,145 filed on Feb. 27, 2015. The current application is filed on Feb. 29, 2016 while Feb. 27, 2016 was on a weekend.

### FIELD OF THE INVENTION

The present invention relates generally to a condom. More specifically, the present invention is an improved system which comprises a condom and a condom wrapper used to help treat erectile dysfunction in a covert manner.

### BACKGROUND OF THE INVENTION

Erectile dysfunction affects millions of men, making the act of sex stressful and difficult to enjoy. Erectile dysfunction makes it harder for men to get and maintain an erection. Traditional condoms can worsen this problem by dulling sensations experienced by the user.

Dealing with erectile dysfunction can be awkward and even embarrassing for men. Solutions for erectile dysfunction can be cumbersome to deal with during intimate moments with a partner. Medications can be effective for some men, but are known to have adverse side effects and can become a hassle in a spontaneous moment. Further, by taking oral medications, often the user reveals that they suffer from erectile dysfunction. This can become an unwanted distraction before and during sex.

Accordingly, there is a present need for an improved condom which can be used to treat erectile dysfunction without becoming a distraction for the user or their partner. The present invention is a system which uses multiple constriction bands and a constriction ring which are integrated into the design of a condom. The constriction bands and the constriction ring blend into the look of the condom so they may go unnoticed during sex. The system includes a condom wrapper which is designed to help aid the user in stretching the constriction bands and the constriction ring so that the condom may be easily put on.

### BRIEF DESCRIPTION OF THE DRAWINGS

FIG. 1 is a front perspective view of the condom.

FIG. 2 is a front view of the condom.

FIG. 3 is a right section view of the condom taken along the line A-A in FIG. 2.

FIG. 4 is a top view of the condom wrapper, wherein the condom and the plurality of pull tabs are shown in dashed lines.

FIG. 5 is an exploded perspective view of the present invention.

FIG. 6 is a top view of the present invention.

FIG. 7 is a front cross-sectional schematic view of the second constriction band, wherein the second constriction band is in a folded configuration.

FIG. 8 is a front view of the condom, wherein the second constriction band is in an unfolded configuration.

FIG. 9 is a cross-sectional schematic view showing lubricant coating internally layered onto the condom, the first constriction band, and the second constriction band.

FIG. 10 is a cross-sectional schematic view showing lubricant coating externally layered onto the condom, the first constriction band, and the second constriction band.

### DETAILED DESCRIPTION OF THE INVENTION

All illustrations of the drawings are for the purpose of describing selected versions of the present invention and are not intended to limit the scope of the present invention.

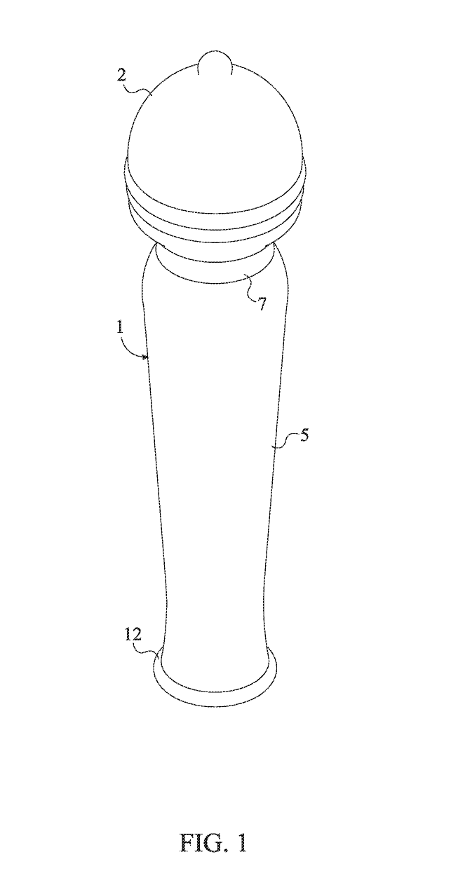

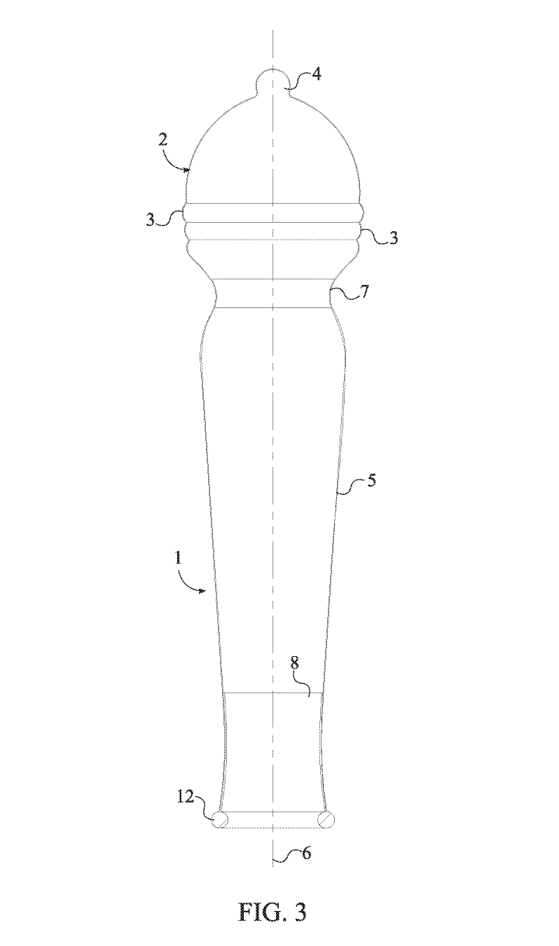

With reference to FIGS. 1-3, the present invention is a system for preventing pregnancy and covertly treating erectile dysfunction. The present invention comprises a condom 1, a first constriction band 7, a second constriction band 8, and a constriction ring 12. The condom 1 is used to prevent pregnancy by capturing semen ejaculated during intercourse. The condom 1 comprises a length-adjustable head 2 and a tubular body 5. The length-adjustable head 2 allows for the condom 1 to be adjusted to ensure user comfort. The tubular body 5 is used to fit over the shaft of the penis and help prevent the condom 1 from slipping during intercourse. The first constriction band 7 is connected adjacent to the tubular body 5 and is used to constrict the glans in order to treat erectile dysfunction. The length-adjustable head 2 is connected adjacent to the first constriction band 7, opposite to the tubular body 5 and helps to ensure that the first constriction band 7 is positioned correctly along the penis. The second constriction band 8 and the constriction ring 12 are also used to treat erectile dysfunction. The second constriction band 8 is connected adjacent to the tubular body 5, opposite to the first constriction band 7. When worn, the second constriction band 8 is positioned about the shaft of the penis. The constriction ring 12 is connected adjacent to the second constriction band 8, opposite to the tubular body 5. This arrangement allows the constriction ring 12 to be positioned about the shaft of the penis, adjacent to the second constriction band 8, while worn.

The length-adjustable head 2, the tubular body 5, the first constriction band 7, the second constriction band 8, and the constriction ring 12 are concentrically aligned about a central axis 6 of the condom 1. This creates a uniform boundary which may be fitted over the penis prior to engaging in sexual intercourse and used to capture ejaculated semen. The first constriction band 7, the second constriction band 8, and the constriction ring 12 are designed to blend in with the look of the condom 1 so that the present invention is virtually indistinguishable from a regular condom. Because erectile dysfunction can be awkward or even embarrassing to talk about, an aim of the present invention is to provide a means of treating erectile dysfunction without revealing that the user experiences erectile dysfunction.

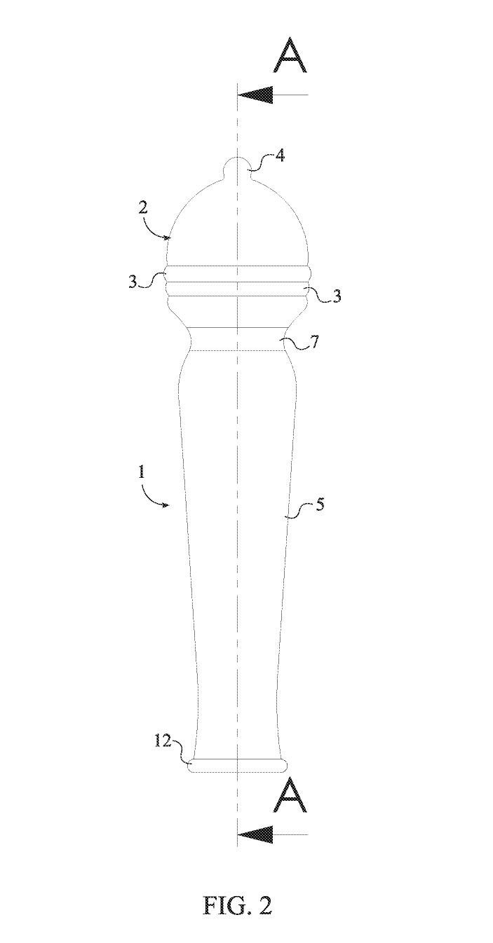

In reference to FIG. 2, the length adjustable head comprises a plurality of pleats 3 and a reservoir tip 4. The plurality of pleats 3 allow the length-adjustable head 2 to be sized for each individual user. The plurality of pleats 3 is positioned offset from the reservoir tip 4 along the length-adjustable head 2. The plurality of pleats 3 may be stretched or unfolded to elongate the length-adjustable head 2. The reservoir tip 4 is used to aid in the capturing of semen, but also helps to prevent the condom 1 from breaking during intercourse.

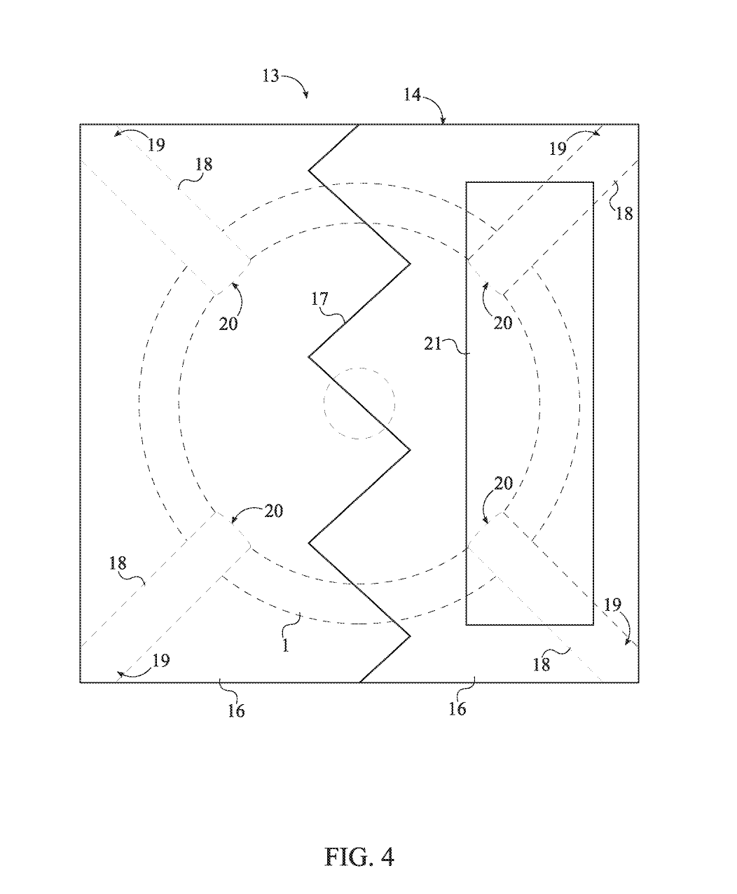

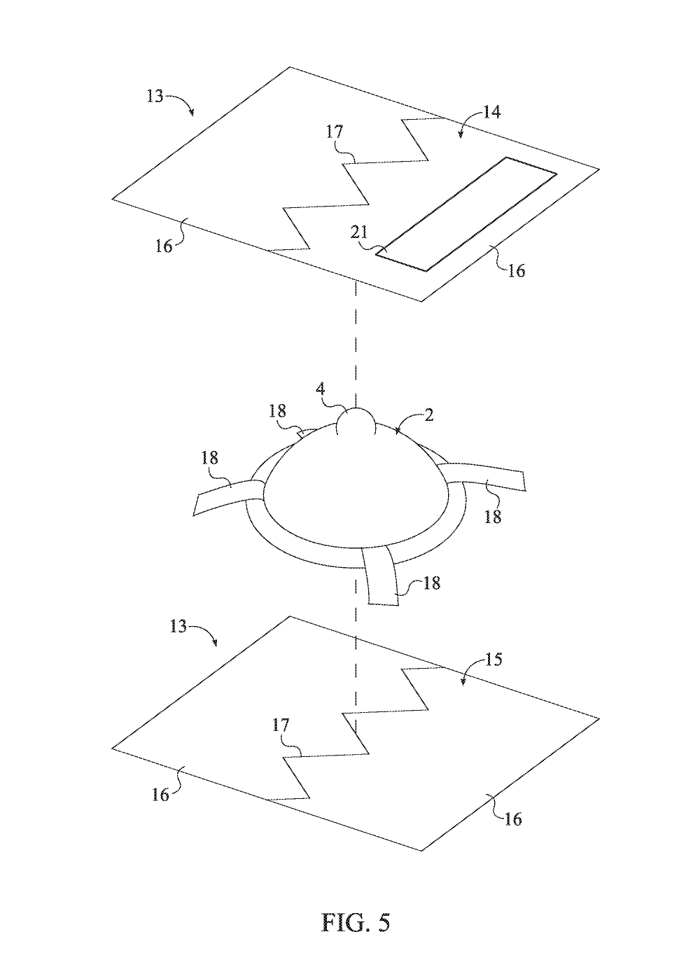

In reference to FIGS. 4-5 the present invention further comprises a condom wrapper 13 which aids the user in putting on the condom 1 and also prevents the condom 1 from being damaged. The condom wrapper 13 comprises a first panel 14 and a second panel 15. The first panel 14 is positioned adjacent to the second panel 15 and is perimetrically connected to the second panel 15. This configuration creates a space between the first panel 14 and the second panel 15. The condom 1, the first constriction band 7, the second constriction band 8, and the constriction ring 12 are positioned in between the first panel 14 and the second panel 15. This arrangement creates a barrier around the condom 1, keeping the condom 1 sterile and intact. Further, this arrangement allows the condom 1 to be coated in lubricants.

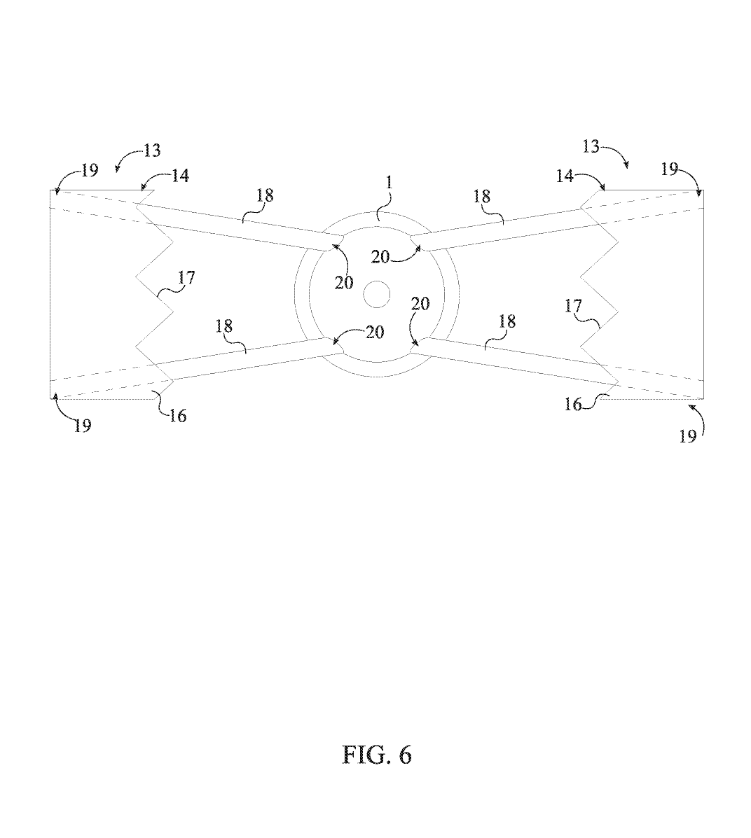

The first panel 14 and the second panel 15 each comprise a pair of assembly pieces 16 and a breakable interface 17. In reference to FIGS. 5-6, the pair of assembly pieces 16 is attached adjacent to each other by the breakable interface 17. When the user wishes to unwrap and put on the condom 1, the first panel 14 and second panel 15 may each be split along the breakable interface 17. This arrangement helps to prevent the condom 1 from being damaged during the process of unwrapping the condom 1. Further, this arrangement makes it easier for the user to unwrap the condom 1.

In order to package individual condoms 1, the second constriction band 8 and the tubular body 5 are rolled about the constriction ring 12 in order to form a rolled configuration with the condom 1, the first constriction band 7, the second constriction band 8, and the constriction ring 12 Like with other condoms, this is done to simplify the process of putting on the condom 1. In reference to FIG. 6, the condom wrapper 13 further comprises a plurality of pull tabs 18 which is distributed about the rolled configuration. The plurality of pull tabs 18 helps to aid the user in putting on the condom 1. A first end 19 for each of the plurality of pull tabs 18 is peripherally connected in between the first panel 14 and the second panel 15. A second end 20 for each of the plurality of pull tabs 18 is rolled about the constriction ring 12 with the second constriction band 8 and the tubular body 5. This is done such that when the user opens the condom wrapper 13, the user is able to stretch the constriction ring 12, the first constriction band 7, the second constriction band 8, and the condom 1 by pulling on the plurality of pull tabs 18. Because the plurality of pull tabs 18 is connected to first panel 14 and the second panel 15, the user does not need to handle each individual pull tab 18. Rather, the user is able to the hold the first panel 14 and the second panel 15 in order to stretch the condom 1. When the condom 1 is completely unrolled onto the penis, the plurality of pull tabs 18 may be removed and the entire condom wrapper 13 may be disposed of. The preferred embodiment of the present invention comprises four pull tabs 18; however, any number of pull tabs 18 may be used in alternative embodiments of the present invention.

In reference to FIG. 5, the present invention further comprises a scent patch 21. The scent patch 21 is mounted onto the condom wrapper 13 and may be scented to enhance the sexual experience of the user or their partner. The scent patch 21 can include, but is not limited to, aphrodisiac scents and pheromones. Further, multiple scents may be included to target the user and their partner.

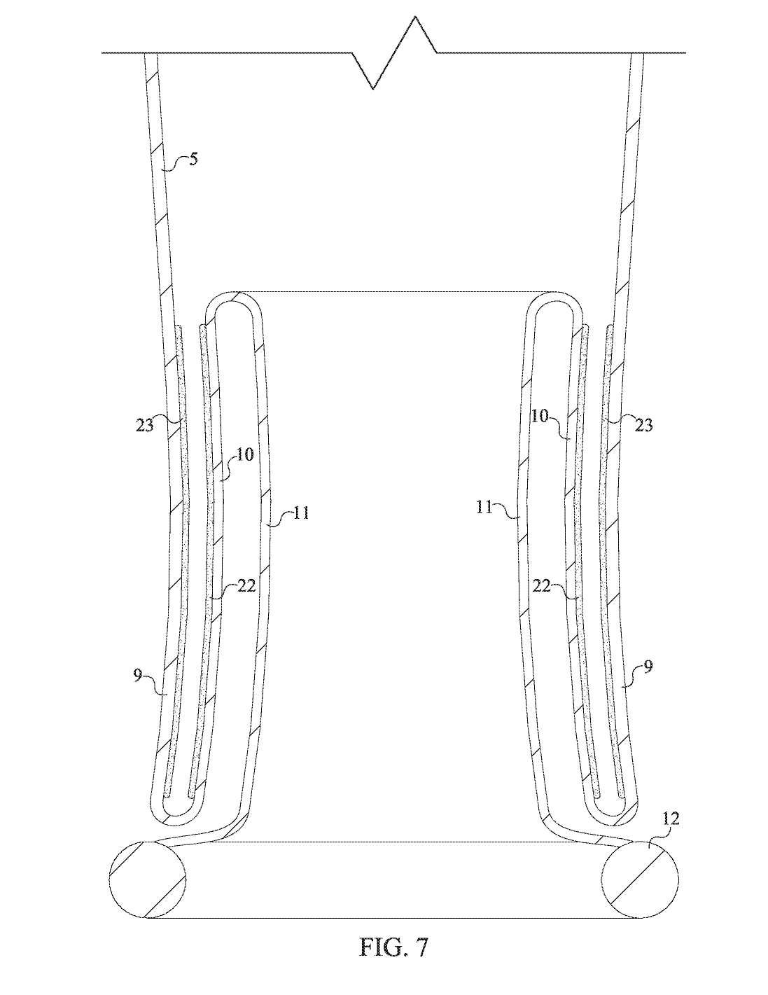

In reference to FIG. 7, the second constriction band 8 comprises a proximal portion 9, an intermediate portion 10, and a distal portion 11. The proximal portion 9, the intermediate portion 10, and the distal portion 11 may be overlapped in order to give strength to the second constriction band 8. The proximal portion 9 is foldably connected adjacent to the tubular body 5. The intermediate portion 10 is foldably connected adjacent to the proximal portion 9, opposite to the tubular body 5. The distal portion 11 is foldably connected adjacent to the intermediate portion 10, opposite to the proximal portion 9. When overlapped, the proximal portion 9, the intermediate portion 10, and the distal portion 11 cause the second constriction band 8 to be three layers thick. The constriction ring 12 is connected adjacent to the distal portion 11, opposite to the intermediate portion 10. Together, the second constriction band 8 and the constriction ring 12 help to prevent blood from flowing out of the penis, resulting in a loss of erection.

In a folded configuration of the second constriction band 8, the intermediate portion 10 is encircled by the proximal portion 9 and the distal portion 11 is encircled by the intermediate portion 10. In addition to strengthening the second constriction band 8, this configuration is also used to hold adhesive materials within the condom 1, without said materials contacting the user's skin. In the preferred embodiment of the present invention, an adhesive coating 22 is internally layered onto the intermediate portion 10 and a non-stick coating 23 is internally layered onto the proximal portion 9. The adhesive coating 22 is used to help seal the condom 1 after use, while the non-stick coating 23 is used to prevent the proximal portion 9 and the intermediate portion 10 from adhering to each other while the second constriction band 8 is in the folded configuration.

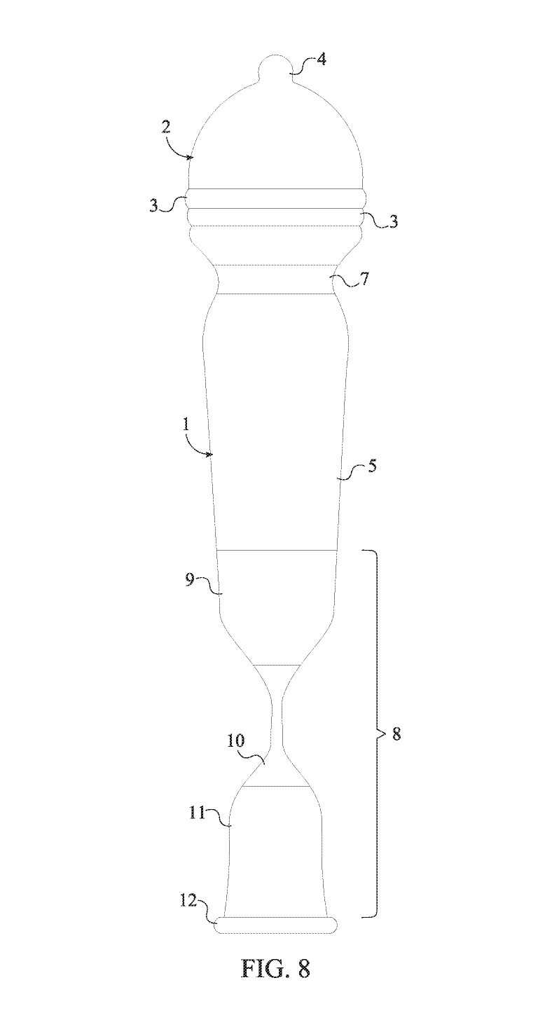

In an unfolded configuration the proximal portion 9, shown in FIG. 8, the intermediate portion 10, and the distal portion 11 are linearly aligned with each other. After using the present invention, the second constriction band 8 may be unfolded by pulling on the constriction ring 12. When the second constriction band 8 unfolds, the adhesive coating 22 is pulled away from the non-stick coating 23. The user may pinch the intermediate portion 10 such that the adhesive coating 22 contacts itself. By doing this, the condom 1 can be sealed easily and without requiring the user to tie the condom 1 in a knot.

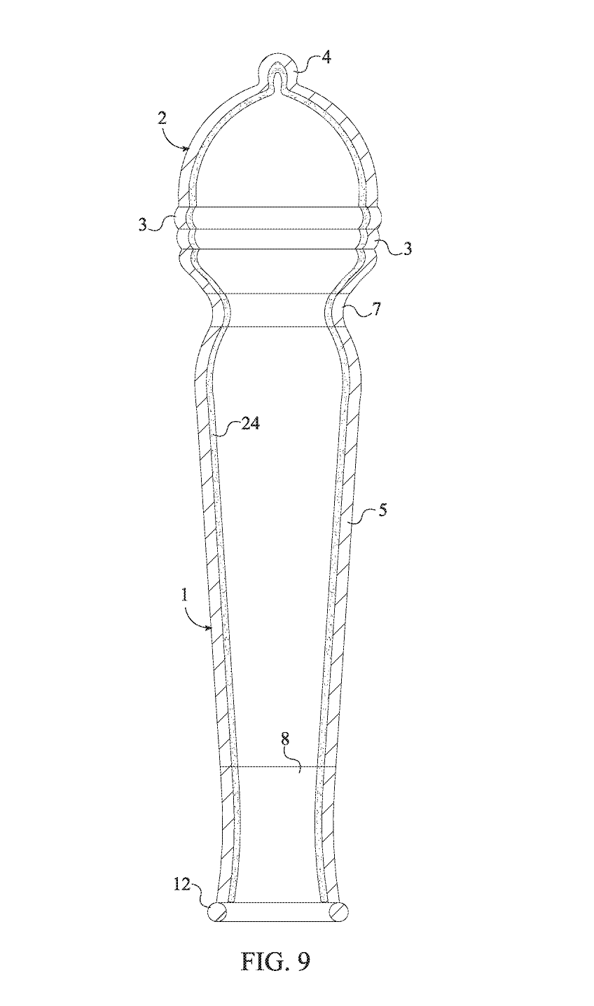

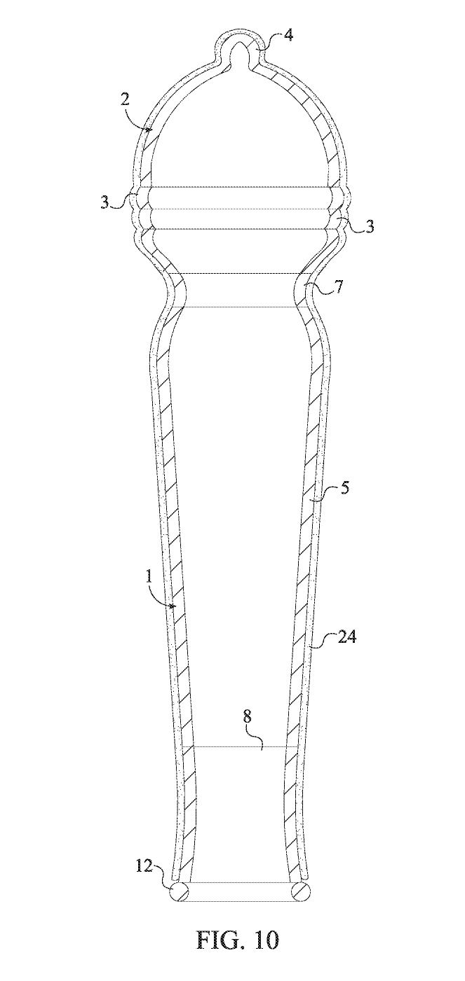

In the preferred embodiment of the present invention, a lubricant coating 24 is internally and/or externally layered onto the condom 1, the first constriction band 7, the second constriction band 8, and the constriction ring 12. This can be seen in FIGS. 9-10. The lubricant coating 24 is used to enhance the sexual experience between the user and their partner. The lubricant coating 24 may include ingredients such as, but not limited to, vasodilation, libido enhancing, and warming agents to further improve the sexual experience between the user and their partner.

Although the invention has been explained in relation to its preferred embodiment, it is to be understood that many other possible modifications and variations can be made without departing from the spirit and scope of the invention as hereinafter claimed.

What is claimed is:

1. A system for preventing pregnancy and covertly treating erectile dysfunction comprises:
a condom;
a first constriction band;
a second constriction band;
a constriction ring;
the condom comprises a length-adjustable head and a tubular body;
the first constriction band being connected adjacent to the tubular body;
the length-adjustable head being connected adjacent to the first constriction band, opposite to the tubular body;
the second constriction band being connected adjacent to the tubular body, opposite to the first constriction band;
the constriction ring being connected adjacent to the second constriction band, opposite to the tubular body; and
the length-adjustable head, the tubular body, the first constriction band, the second constriction band, and the constriction ring being concentrically aligned about a central axis of the condom.

2. The system for preventing pregnancy and covertly treating erectile dysfunction as claimed in claim 1 comprises:
the length-adjustable head comprises a plurality of pleats and a reservoir tip; and
the plurality of pleats being positioned offset from the reservoir tip along the length-adjustable head.

3. The system for preventing pregnancy and covertly treating erectile dysfunction as claimed in claim 1 comprises:
a condom wrapper;
the condom wrapper comprises a first panel and a second panel;
the first panel being positioned adjacent to the second panel;
the first panel being perimetrically connected to the second panel; and
the condom, the first constriction band, the second constriction band, and the constriction ring being positioned in between the first panel and the second panel.

4. The system for preventing pregnancy and covertly treating erectile dysfunction as claimed in claim 3 comprises:
the first panel and the second panel each comprise a pair of assembly pieces and a breakable interface; and
the pair of assembly pieces being attached adjacent to each other by the breakable interface.

5. The system for preventing pregnancy and covertly treating erectile dysfunction as claimed in claim 3 comprises:
wherein the second constriction band and the tubular body being rolled about the constriction ring in order to form a rolled configuration with the condom, the first constriction band, the second constriction band, and the constriction ring;
the condom wrapper further comprises a plurality of pull tabs;
the plurality of pull tabs being distributed about the rolled configuration;
a first end for each of the plurality of pull tabs being peripherally connected in between the first panel and the second panel; and
a second end for each of the plurality of pull tabs being rolled about the constriction ring with the second constriction band and the tubular body.

6. The system for preventing pregnancy and covertly treating erectile dysfunction as claimed in claim 3 comprises:
a scent patch; and
the scent patch being mounted onto the condom wrapper.

7. The system for preventing pregnancy and covertly treating erectile dysfunction as claimed in claim 1 comprises:
the second constriction band comprises a proximal portion, an intermediate portion, and a distal portion;
the proximal portion being foldably connected adjacent to the tubular body;
the intermediate portion being foldably connected adjacent to the proximal portion, opposite to the tubular body;
the distal portion being foldably connected adjacent to the intermediate portion, opposite to the proximal portion; and
the constriction ring being connected adjacent to the distal portion, opposite to the intermediate portion.

8. The system for preventing pregnancy and covertly treating erectile dysfunction as claimed in claim 7 comprises:
wherein the second constriction band is in a folded configuration;
the intermediate portion being encircled by the proximal portion; and
the distal portion being encircled by the intermediate portion.

9. The system for preventing pregnancy and covertly treating erectile dysfunction as claimed in claim 7 comprises:
wherein the second constriction band is in an unfolded configuration; and
the proximal portion, the intermediate portion, and the distal portion being linearly aligned with each other.

10. The system for preventing pregnancy and covertly treating erectile dysfunction as claimed in claim 7, wherein an adhesive coating is internally layered onto the intermediate portion.

11. The system for preventing pregnancy and covertly treating erectile dysfunction as claimed in claim 7, wherein a non-stick coating is internally layered onto the proximal portion.

12. The system for preventing pregnancy and covertly treating erectile dysfunction as claimed in claim 1, wherein a lubricant coating is internally layered onto the condom, the first constriction band, and the second constriction band.

13. The system for preventing pregnancy and covertly treating erectile dysfunction as claimed in claim 1, wherein a lubricant coating is externally layered onto the condom, the first constriction band, and the second constriction band.

14. A system for preventing pregnancy and covertly treating erectile dysfunction comprises:
a condom;
a first constriction band;
a second constriction band;
a constriction ring;
the condom comprises a length-adjustable head and a tubular body;
the length-adjustable head comprises a plurality of pleats and a reservoir tip;
the first constriction band being connected adjacent to the tubular body;
the length-adjustable head being connected adjacent to the first constriction band, opposite to the tubular body;
the second constriction band being connected adjacent to the tubular body, opposite to the first constriction band;
the constriction ring being connected adjacent to the second constriction band, opposite to the tubular body;
the length-adjustable head, the tubular body, the first constriction band, the second constriction band, and the constriction ring being concentrically aligned about a central axis of the condom;
the plurality of pleats being positioned offset from the reservoir tip along the length-adjustable head; and
a lubricant coating being externally layered onto the condom, the first constriction band, and the second constriction band.

15. The system for preventing pregnancy and covertly treating erectile dysfunction as claimed in claim 14 comprises:
a condom wrapper;
the condom wrapper comprises a first panel, a second panel, and a plurality of pull tabs;
the first panel and the second panel each comprise a pair of assembly pieces and a breakable interface;
the first panel being positioned adjacent to the second panel;
the first panel being perimetrically connected to the second panel;
the condom, the first constriction band, the second constriction band, and the constriction ring being positioned in between the first panel and the second panel;
the pair of assembly pieces being attached adjacent to each other by the breakable interface;
wherein the second constriction band and the tubular body being rolled about the constriction ring in order to form a rolled configuration with the condom, the first constriction band, the second constriction band, and the constriction ring;
the plurality of pull tabs being distributed about the rolled configuration;
a first end for each of the plurality of pull tabs being peripherally connected in between the first panel and the second panel; and
a second end for each of the plurality of pull tabs being rolled about the constriction ring with the second constriction band and the tubular body.

16. The system for preventing pregnancy and covertly treating erectile dysfunction as claimed in claim 15 comprises:
a scent patch; and
the scent patch being mounted onto the condom wrapper.

17. The system for preventing pregnancy and covertly treating erectile dysfunction as claimed in claim 14 comprises:
the second constriction band comprises a proximal portion, an intermediate portion, and a distal portion;
the proximal portion being foldably connected adjacent to the tubular body;
the intermediate portion being foldably connected adjacent to the proximal portion, opposite to the tubular body;
the distal portion being foldably connected adjacent to the intermediate portion, opposite to the proximal portion; and
the constriction ring being connected adjacent to the distal portion, opposite to the intermediate portion.

18. The system for preventing pregnancy and covertly treating erectile dysfunction as claimed in claim 17 comprises:
wherein the second constriction band is in a folded configuration;
the intermediate portion being encircled by the proximal portion; and
the distal portion being encircled by the intermediate portion.

19. The system for preventing pregnancy and covertly treating erectile dysfunction as claimed in claim 17 comprises:
wherein the second constriction band is in an unfolded configuration; and
the proximal portion, the intermediate portion, and the distal portion being linearly aligned with each other.

20. The system for preventing pregnancy and covertly treating erectile dysfunction as claimed in claim 17 comprises:
an adhesive coating being internally layered onto the intermediate portion; and
a non-stick coating being internally layered onto the proximal portion.
